(19) United States
(12) Patent Application Publication
(10) Pub. No.: US 2005/0147561 A1
(43) Pub. Date: Jul. 7, 2005

(54) Method for identifying pesticidal active compounds

(76) Inventors: Thomas Schulte, Koln (DE); Ulrich Ebbinghaus-Kintscher, Dortmund (DE); Peter Lummen, Idstein (DE); Rudiger Fischer, Pulheim (DE); Christian Funke, Leichlingen (DE)

Correspondence Address: BAYER CROPSCIENCE LP;Patent Department, 100 BAYER ROAD, PITTSBURGH, PA, 15205-9741, US

(21) Appl. No.: 10/956,505

(22) Filed: Oct. 1, 2004

(30) Foreign Application Priority Data
Oct. 10, 2003 (DE) 10347089.1
Dec. 4, 2003 (DE) 10356553.1

(51) Int. Cl. A61K 49/00; A01N 37/34; A01N 37/10; A01N 37/18

(52) U.S. Cl. 424/9.2; 514/616; 514/522

(57) ABSTRACT

The present invention relates to a method for identifying pesticidally active substances. The present invention relates in particular to a binding assay with labelled phthalic diamides on membrane preparations and the displacement of these labelled phthalic diamides by unlabelled test substances.

### BACKGROUND OF THE INVENTION

1. Field of the Invention

The present invention relates to a method for identifying pesticidal active compounds.

2. Description of the Related Art

Compounds from the class of the phthalic diamides (cf. EP-A-0 919 542, EP-A-1 006 107, WO 01/00575, WO 01/00599, WO 01/46124, JP 2001-33 555 9, WO 01/02354, WO 01/21576, WO 02/088074, WO 02/088075, WO 02/094765, WO 02/094766, WO 02/062807) are highly potent pesticides. The mechanism of action of these compounds is not yet in the public domain and constitutes a new active principle. A high-throughput screening method for identifying active compounds which are subject to the same mechanism is therefore very valuable.

### SUMMARY OF THE INVENTION

The object of the present invention is therefore to provide a method by means of which pesticidal active compounds which operate by acting on the same target as that of the phthalic diamides can be identified.

The object is achieved by providing a method which is characterized in that the displacement of a i) labelled compound as shown in the general Formula (I) hereinbelow or ii) of a labelled compound which binds to the same binding site as a compound of the general Formula (I) on membrane preparations or cell lines containing the molecular target of the compounds of the general formula 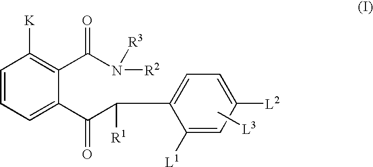
in which
K represents halogen, cyano, alkyl, halogenoalkyl, alkoxy or halogenoalkoxy
R¹, R², R³ in each case independently of one another represent hydrogen, cyano, C₃-C₈-cycloalkyl which is optionally substituted by halogen, or a group of the formula M¹-Qₖ in which
   M¹ represents optionally substituted alkylene, alkenylene or alkynylene,
   Q represents hydrogen, halogen, cyano, nitro, halogenoalkyl, in each case optionally substituted C₃-C₈-cycloalkyl, alkylcarbonyl or alkoxycarbonyl, in each case optionally substituted phenyl, hetaryl or a group T-R⁴ in which
      T represents —O—, —S(O)ₘ— or 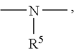
R⁴ represents hydrogen, in each case optionally substituted alkyl, alkenyl, alkynyl, cycloalkyl, cycloalkyl-alkyl, alkoxyalkyl, alkylcarbonyl, alkoxycarbonyl, phenyl, phenylalkyl, phenylalkoxy, hetaryl, hetarylalkyl,
R⁵ represents hydrogen, in each case optionally substituted alkylcarbonyl, alkoxycarbonyl, phenylcarbonyl or phenylalkoxycarbonyl,
   k represents the numbers 1 to 4,
   m represents the numbers 0 to 2, or
R¹ and R² together form optionally substituted four- to seven-membered rings which can optionally be interrupted by hetero atoms,
L¹ and L³ independently of one another represent hydrogen, halogen, cyano or in each case optionally substituted alkyl, alkoxy, alk-S(O)ₘ—, phenyl, phenoxy or hetaryloxy,
L² represents hydrogen, halogen, cyano, in each case optionally substituted alkyl, alkenyl, alkynyl, halogenoalkyl, cycloalkyl, phenyl, hetaryl or the group M²-R⁶ in which
   M² represents —O— or —S(O)ₘ—
   R⁶ represents in each case optionally substituted alkyl, alkenyl, alkynyl, cycloalkyl, phenyl or hetaryl, or
L¹ and L³ or L¹ and L² optionally together form an optionally substituted five- to six-membered ring which can optionally be interrupted by hetero atoms is measured.

### BRIEF DESCRIPTION OF THE DRAWINGS

FIG. 1 is a plot illustrating the inhibition of the specific binding of compound 1 by the test substance (Compound 2) as a function of concentration.

### DETAILED DESCRIPTION OF THE INVENTION

As indicated above, the object of the present invention is to provide a method by means of which pesticidal active compounds which operate by acting on the same target as that of the phthalic diamides can be identified.

Also as indicated above, the object is achieved by providing a method which is characterized in that the displacement of a i) labelled compound as shown in the general Formula (I) hereinbelow or ii) of a labelled compound which binds to the same binding site as a compound of the general Formula (I) on membrane preparations or cell lines containing the molecular target of the compounds of the general Formula (I) 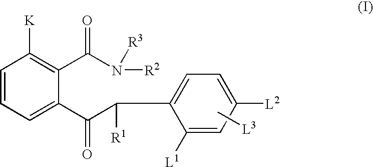
in which
K represents halogen, cyano, alkyl, halogenoalkyl, alkoxy or halogenoalkoxy,
R¹, R², R³ in each case independently of one another represent hydrogen, cyano, C₃-C₈-cycloalkyl which is optionally substituted by halogen, or a group of the formula M¹-Qₖ in which
   M¹ represents optionally substituted alkylene, alkenylene or alkynylene,
   Q represents hydrogen, halogen, cyano, nitro, halogenoalkyl, in each case optionally substituted C₃-C₈-cycloalkyl, alkylcarbonyl or alkoxycarbonyl, in each case optionally substituted phenyl, hetaryl or a group T-R⁴ in which
      T represents —O—, —S(O)ₘ— or 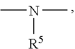
R⁴ represents hydrogen, in each case optionally substituted alkyl, alkenyl, alkynyl, cycloalkyl, cycloalkyl-alkyl, alkoxyalkyl, alkylcarbonyl, alkoxycarbonyl, phenyl, phenylalkyl, phenylalkoxy, hetaryl, hetarylalkyl,
R⁵ represents hydrogen, in each case optionally substituted alkylcarbonyl, alkoxycarbonyl, phenylcarbonyl or phenylalkoxycarbonyl,
   k represents the numbers 1 to 4,
   m represents the numbers 0 to 2, or
R¹ and R² together form optionally substituted four- to seven-membered rings which can optionally be interrupted by hetero atoms,
L¹ and L³ independently of one another represent hydrogen, halogen, cyano or in each case optionally substituted alkyl, alkoxy, alk-S(O)ₘ—, phenyl, phenoxy or hetaryloxy,
L² represents hydrogen, halogen, cyano, in each case optionally substituted alkyl, alkenyl, alkynyl, halogenoalkyl, cycloalkyl, phenyl, hetaryl or the group M²-R⁶ in which
   M² represents —O— or —S(O)ₘ—,
   R⁶ represents in each case optionally substituted alkyl, alkenyl, alkynyl, cycloalkyl, phenyl or hetaryl, or
L¹ and L³ or L¹ and L² optionally together form an optionally substituted five- to six-membered ring which can optionally be interrupted by hetero atoms is measured.

Preferred substituents or ranges of the radicals specified in the general Formula (I) are illustrated hereinbelow.
K preferably represents fluorine, chlorine, bromine, iodine, cyano, C₁-C₆-alkyl, C₁-C₆-halogenoalkyl, C₁-C₆-alkoxy or C₁-C₆-halogenoalkoxy.
R¹, R², R³ in each case independently of one another preferably represent hydrogen, cyano, C₃-C₆-cycloalkyl which is optionally substituted by halogen, or a group of the formula M¹-Qₖ, in which
   M¹ represents C₁-C₈-alkylene, C₃-C₆-alkenylene or C₃-C₆-alkynylene,
   Q represents hydrogen, halogen, cyano, nitro, halogenoalkyl, or represents C₃-C₈-cycloalkyl which is optionally substituted by fluorine, chlorine, C₁-C₆-alkyl or C₁-C₆-alkoxy and in which one or two ring members which are not directly adjacent are optionally replaced by oxygen and/or sulphur, or represents C₁-C₆-alkylcarbonyl or C₁-C₆-alkoxycarbonyl, each of which is optionally substituted by halogen, or represents phenyl or hetaryl having five to six ring atoms, each of these phenyl or hetaryl rings optionally being substituted by halogen, C₁-C₆-alkyl, C₁-C₆-halogenoalkyl, C₁-C₆-alkoxy, C₁-C₆-halogenoalkoxy, cyano or nitro, or represents a group T-R⁴ in which
T represents —O—, —S(O)ₘ— or 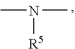
R⁴ represents hydrogen, or represents C₁-C₈-alkyl, C₃-C₈-alkenyl, C₃-C₈-alkynyl, C₃-C₈-cycloalkyl, C₃-C₈-cycloalkyl-C₁-C₂-alkyl, C₁-C₆-alkylcarbonyl, C₁-C₆-alkoxycarbonyl, each of which is optionally substituted by fluorine and/or chlorine, or represents phenyl, C₁-C₄-phenylalkyl, C₁-C₄-phenylalkyloxy, hetaryl or hetarylalkyl, each of which is optionally monosubstituted to tetrasubstituted by halogen, C₁-C₆-alkyl, C₁-C₆-alkoxy, C₁-C₄-halogenoalkyl, C₁-C₄-halogenoalkoxy, nitro or cyano, hetaryl encompassing 5 to 6 ring atoms and representing in particular furanyl, pyridyl, imidazolyl, triazolyl, pyrazolyl, pyrimidyl, thiazolyl or thienyl,
R⁵ represents hydrogen, or represents C₁-C₆-alkylcarbonyl, C₁-C₆-alkoxycarbonyl, each of which is optionally substituted by fluorine and/or chlorine, or represents phenylcarbonyl or phenyl-C₁-C₄-alkyloxycarbonyl, each of which is optionally monosubstituted to tetrasubstituted by halogen, C₁-C₆-alkyl, C₁-C₆-alkoxy, C₁-C₄-halogenoalkyl, C₁-C₄-halogenoalkoxy, nitro or cyano,
   k represents the numbers 1 to 3 and
   m represents the numbers 0 to 2.
R¹ and R² can also form a five- to six-membered ring which can optionally be interrupted by an oxygen or sulphur atom.
L¹ and L³ independently of one another preferably represent hydrogen, fluorine, chlorine, bromine, iodine, cyano, C₁-C₆-alkyl, C₁-C₄-halogenoalkyl, C₁-C₆-alkoxy, C₁-C₄-halogenoalkoxy, C₁-C₄-alkyl-S(O)ₘ—, C₁-C₄-haloalkyl-S(O)ₘ—, or represent phenyl, phenoxy, pyridyloxy, thiazolyloxy or pyrimidyloxy, each of which is optionally monosubstituted to trisubstituted by fluorine, chlorine, bromine, C₁-C₆-alkyl, C₁-C₆-alkoxy, C₁-C₄-halogenoalkyl, C₁-C₄-halogenoalkoxy, cyano or nitro.
L² preferably represents hydrogen, fluorine, chlorine, bromine, iodine, cyano, or represents C₁-C₁₀-alkyl, C₂-C₁₀-alkenyl, C₂-C₆-alkynyl, each of which is optionally substituted by fluorine and/or chlorine, or represents C₃-C₆-cycloalkyl, which is optionally substituted by fluorine or chlorine, or represents phenyl, pyridyl, thienyl, pyrimidyl or thiazolyl, each of which is optionally monosubstituted to trisubstituted by fluorine, chlorine, bromine, C₁-C₆-alkyl, C₁-C₆-alkoxy, C₁-C₄-halogenoalkyl, C₁-C₄-halogenoalkoxy, cyano or nitro, or represents a group M²-R⁶ in which
   M² represents —O— or —S(O)ₘ— and
   R⁶ represents C₁-C₈-alkyl, C₂-C₈-alkenyl, C₃-C₆-alkynyl or C₃-C₆-cycloalkyl, each of which is optionally substituted by fluorine and/or chlorine, or represents phenyl, pyridyl, pyrimidyl or thiazolyl, each of which is optionally monosubstituted to trisubstituted by fluorine, chlorine, bromine, C₁-C₆-alkyl, C₁-C₆-alkoxy, C₁-C₄-halogenoalkyl, C₁-C₄-halogenoalkoxy, cyano or nitro.
L¹ and L³ or L² and L₃ can also in each case together form a five- to six-membered ring which is optionally substituted by fluorine and/or C₁-C₂-alkyl which, if appropriate, can be interrupted by one or two oxygen atoms.
K especially preferably represents chlorine, bromine or iodine.
R¹, R², R³ in each case independently of one another especially preferably represent hydrogen or a group of the formula M¹-Qₖ, in which
   M¹ represents C₁-C₈-alkylene, C₃-C₆-alkenylene or C₃-C₆-alkynylene,
   Q represents hydrogen, fluorine, chlorine, cyano, trifluoromethyl, C₃-C₆-cycloalkyl or a group T-R⁴ in which
      T represents —O— or —S(O)ₘ—,
      R⁴ represents hydrogen, or represents C₁-C₆-alkyl, C₃-C₆-alkenyl, C₃-C₆-alkynyl or C₃-C₆-cycloalkyl, each of which is optionally monosubstituted to trisubstituted by fluorine and/or chlorine,
   k represents the numbers 1 to 3 and
   m represents the numbers 0 to 2.
L¹ and L³ independently of one another especially preferably represent hydrogen, fluorine, chlorine, bromine, iodine, cyano, C₁-C₄-alkyl, C₁-C₂-halogenoalkyl, C₁-C₄-alkoxy, C₁-C₂-halogenoalkoxy, or represent phenyl or phenoxy, each of which is optionally monosubstituted or disubstituted by fluorine, chlorine, bromine, C₁-C₄-alkyl, C₁-C₄-alkoxy, C₁-C₂-halogenoalkyl, C₁-C₂-halogenoalkoxy, cyano or nitro.
L² especially preferably represents hydrogen, fluorine, chlorine, bromine, iodine, cyano, or represents C₁-C₆-alkyl, C₂-C₆-alkenyl, C₂-C₆-alkynyl, C₃-C₆-cycloalkyl, each of which is optionally monosubstituted to tridecasubstituted by fluorine and/or chlorine, or represents a group M²-R⁶ in which
   M² represents —O— or —S(O)ₘ— and
   R⁶ represents C₁-C₆-alkyl, C₂-C₆-alkenyl, C₂-C₆-alkynyl or C₃-C₆-cycloalkyl, each of which is optionally monosubstituted to tridecasubstituted by fluorine and/or chlorine, or represents phenyl or pyridyl, each of which is optionally monosubstituted to disubstituted by fluorine, chlorine, bromine, C₁-C₄-alkyl, C₁-C₄-alkoxy, trifluoromethyl, difluoromethoxy, trifluoromethoxy, cyano or nitro.

The compound of the general Formula (I) is very especially preferably the following compound:

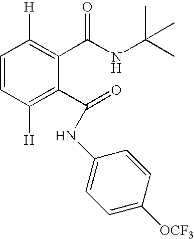

The compounds of the general Formula I are known compounds which are described in, or encompassed by, the following publications (cf. EP-A-0 919 542, EP-A-1 006 107, WO 01/00575, WO 01/00599, WO 01/46124, JP 2001-335559, WO 01/02354, WO 01/21576, WO 02/088074, WO 02/088075, WO 02/094765, WO 02/094766, WO 02/062807). They can be prepared by the methods described in these publications.

The present invention therefore relates to binding assays with labelled phthalic diamides on membrane preparations of suitable organisms or cell lines containing the molecular target of the phthalic diamides, and the displacement of these labelled phthalic diamides by unlabelled test substances. In principle, such test compounds or candidate compounds can be any compound which may belong to very different types of compound. The skilled worker is familiar with a multiplicity of sources which contain compounds which are suitable for the method according to the invention. Sources which are suitable in principle are, for example, all feasible substance libraries; libraries of low-molecular-weight compounds, in particular small organochemical compounds, are preferred. If appropriate, a phthalic diamide compound which binds to the target with high specificity can be displaced from the binding site by the test substance. Labelling is generally understood as meaning a modification, in the molecule to be displaced, which facilitates detection. Examples are radio labels, fluorescent labels or luminescent labels.

The present invention is based on the finding that the molecular target of the phthalic diamides is membrane-bound and can be targeted by a binding strip displacement assay.

The following text describes the theoretical background of the method according to the invention. A compound of the general Formula (I) is also referred to as “ligand”:

The Simple Occupation Theory describes the reversible ligand-receptor binding in the form of a first-order velocity law. In binding experiments, the ligand is generally present in a large excess relative to the receptor concentration, so that the concentration of the free ligand can be considered as constant in a first approximation. The binding kinetic thus only depends on the receptor concentration (pseudo-first-order velocity law).

The Law of Mass Action stipulates that the position of the binding equilibrium is described by the dissociation constant (reciprocal of the association constant) K_{d}=Kₐ⁻¹=k_{off}/kₒₙ=[R]·[L]/[RL]: k_{off} is the dissociation rate, kₒₙ is the association rate, [R] is the concentration of the free receptors, [L] is the concentration of the unbound ligand, [RL] is the concentration of the receptor-ligand complex. It is proportional to the free energy of the binding: ΔG₀ =R·T·ln K_{d}; ΔG₀ is the Gibbs free energy of the binding, R is the universal gas constant: 8.315 J mol⁻¹ K⁻¹, T is the absolute temperature (in Kelvin) [Repke, H., C. Liebmann; Membranrezeptoren und ihre Effektorsysteme [membrane receptors and their effector systems]; VCH (Weinheim), 1987]. Accordingly, a K_{d} which is reduced by a factor of 10 means a gain of −5.71 kJ mol⁻¹ K⁻¹ of free binding energy.

The concentration of the specifically bound ligand [RL] is determined in saturation-binding experiments as a function of the ligand concentration [L] at constant receptor concentration. To determine the concentration, the ligand is labelled for example by incorporating a radioisotope or by substitution with a fluorophore. Plotting [RL] versus [L] gives a typical saturation curve. In the event that [L]=K_{d}, [RL]=½ [Rₜ]; [Rₜ], the total receptor concentration, is the sum of the concentration of the free receptors [R] and the concentration of the receptor-ligand complexes [RL]. In the presence of saturating ligand concentrations, the concentration of the receptor-ligand complexes corresponds to the total concentration of the receptors, frequently also referred to as maximum binding (Bₘₐₓ).

The kinetic equilibrium parameters K_{d} and Bₘₐₓ are calculated by fitting the data to a hyperbolic model with the aid of suitable PC programs, for example EBDA/LIGAND (BioSoft) or SigmaPlot (SPSS).

Competition experiments describe the effect of unlabelled test substances (referred to as inhibitor, I) on the binding of the labelled ligand. In the simplest case, the inhibitor competes with the labelled ligand for the binding site which they share.

The reduction of the specific binding of the labelled ligand is determined experimentally as a function of the inhibitor concentration. Plotting the specific binding versus the logarithm of the inhibitor concentration gives a characteristic sigmoidal curve whose point of inflection characterizes the inhibitor concentration at which 50% of the ligand binding is inhibited (IC₅₀ value). The dissociation constant of the inhibitor Kᵢ has the following relationship with the ligand concentration [L] and the dissociation constant of the labelled ligand K_{d}: Kᵢ=IC₅₀/(1+[L]/K_{d}) [Cheng, Y., W. H. Prusoff; Biochem. Pharmacol. 22, 3099-3108 (1973)].

Receptor ligands with novel structures and properties are identified with the aid of competition screenings which are automatable in principle and can thus be carried out in a high-throughput method.

The method according to the invention can be carried out using the techniques described hereinbelow.

Radiolabelling of the ligand (i.e. a compound of the general Formula (I)) and scintillation measurement:

The incorporation of radioisotopes was and is the most frequent labelling technique for receptor ligands in affinity-based high-throughput screenings. Tritium (³H) and ¹²⁵I meet the requirement for high specific radioactivity of the ligands (>30 Ci mmol⁻¹), which permits a sufficiently high signal-to-noise ratio, even in nanomolar assay concentrations. Tritiation in particular does not alter the biochemical properties of the ligands and is relatively simple and inexpensive to perform by radiochemical standard methods (direct incorporation, halogen/tritium substitution, catalytic hydrogenation). An advantage of tritiation over iodination in view of the radiochemical stability is the long half-life (12.3 years; by comparison: half-life of ¹²⁵I amounts to 60 days).

All conventional radioactive binding tests share the feature that they require the separation of bound and free ligands. This can be achieved by equilibrium dialysis, centrifugation, gel filtration or filtration through suitable membranes [Keen, M. (ed.); Receptor Binding Techniques; Meth. Molecular Biology, Vol. 106, Humana Press, Totowa, N.J. (1999)].

An example of a technique among the abovementioned methods where miniaturization (microtiter plate format) and automation (robot) are meaningful is the filtration technique.

In view of the high-throughput screening of extensive substance libraries, homogeneous assay methods were developed where the separation of bound and free ligands can be dispensed with. The system sold by Amersham Biosciences under the name Scintillation Proximity Assay (SPA) is widely used. The receptors, solubilized, membrane-bound, prepared from native tissue or expressed recombinantly, are immobilized on spherical particles containing a solid-phase scintillator. If the radioligand binds, the β particles (electrons) emitted generate a light signal in the scintillator which is measured with a B counter [Nelson, N.; Anal. Biochem. 165, 287-293 (1987), Alouani, S.; Meth. Mol. Biol. 138, 135-141 (2000)].

Examples of receptors where the SPA method has been employed for research problems or screening are detailed hereinbelow: IP₃ receptor [Patel, S., et al.; Br. J. Pharmacol. 115, Proc. Suppl. 35p. (1995)], 5-HTle receptor [Kahl, S. D., et al.; J. Biomol. Screening 2, 33-40 (1995)], ligand screening with FKBP-12 [Graziani, F., et al.; J. Biomol. Screening 4, 3-7 (1999)], α-adrenergic receptors [Gobel, J., et al.; J. Pharmacol. Toxicol. Meth. 42, 237-244 (1999)], GABA_{B} receptors [Urwyler, S., et al.; Mol. Pharmacol. 60, 963-971 (2001)].

The FlashPlates (Perkin Elmer/NEN), where the receptors are immobilized in microtiter plate cavities, work in a similar manner. The β particles emitted by bound radioligands excite the solid-phase scintillator in the microtiter plates. Among the various fluorescence-based detection methods, fluorescence polarization (FP) is particularly suitable for binding assays. Intrinsically fluorescent ligands, or ligands which are labelled with a fluorophore, are excited with linearly polarized light. The anisotropism of the light emitted by the freely diffusing, excited ligand molecules is substantially lower since the molecules perform gyratory movements and oscillations during the life of the excited state. In contrast, the rotation of receptor-bound ligands is greatly limited so that their fluorescence has a substantially higher degree of polarization (anisotropism), which corresponds approximately to that of the excited radiation. Measuring the fluorescence polarization makes possible a direct determination of the concentrations of the bound and of the free ligands [Sundberg, S. S.; Curr. Opinion Biotechnol. 11, 47-53 (2000)].

The literature describes FP-based high-throughput binding assays for soluble receptors, for example steroid receptors [Parker, G. J., et al.; J. Biomol. Screen. 5, 77-88 (2000)] and membrane-bound receptors, for example G-protein-coupled receptors [Allen, M., et al.; J. Biomol. Screen. 5, 63-70 (2000), Banks, P., et al.; J. Biomol. Screen. 5, 159-168 (2000)].

FP binding assays are generally characterized by high sensititivy in the sub-nanomolar concentration range and by a good signal-to-noise ratio. However, fluorescent test substances may interfere with the signal.

The following text describes how the biological material, in particular the membrane preparations, for the method according to the invention can be obtained.

Animal tests, in particular agronomically relevant harmful organisms such as, for example, plant- or seed-damaging insects (in particular from the orders Homoptera, Lepidoptera and Coleoptera), arachnids (in particular Acarina) and plant-parasitic nematodes, but also model insects which have been studied thoroughly at the biochemical or molecular-biological level, such as fruit flies (Drosophila melangaster), house flies (Musca domestica), American cockroaches (Periplaneta americana) and grasshoppers and locusts (Locusta spp., Schistocerca spp.), constitute a suitable biological starting material for the preparation of biological membranes containing phthalic diamide receptors.

To carry out the method according to the invention it is possible, for example, to homogenize prepared insect nerve or muscle tissue in a biological standard buffer at neutral pH. Membranes containing phthalic diamide receptors can be obtained by differential centrifugation. Intact cells, large cell membrane fragments and nuclei are separated in a first centrifugation step at approximately 800×g.

Mitochondria and a large part of the cell membranes are removed from the homogenate of approximately 8000×g. Membranes containing phthalic diamide receptors are sedimented at 10⁵×g, suspended in a suitable biological buffer and stored at −20 to −80° C. until use.

Membrane preparations which have been prepared starting from adult organisms of the species Heliothis virescens or their L5 instars, or the species Musca domestica, are preferably used for the method according to the invention.

Membranes from host cells, in particular CHO cells, which are transiently or stably transfected with a cDNA based on the Drosophila melanogaster CG10844 gene from the reference [Xu, X., et al.; Biophys. J. 78, 1270-1281 (2000)] or on the corresponding gene of other organisms can be employed in a further embodiment of the method according to the invention. Providing the cDNA in question and expressing it in suitable host cells is within the ability of the skilled worker. The gene products in question constitute the molecular target of the compounds of the general Formula (I). Cells which do not express the molecular target of the phthalic diamides, such as, for example, CHO, HEK-293, 3T3, SF9 or Schneider 2 cells, are preferably used as host cells.

### EXAMPLES

### Example 1

Preparation of a Labelled Compound of the General Formula (I) by Substituting Tritium for Hydrogen

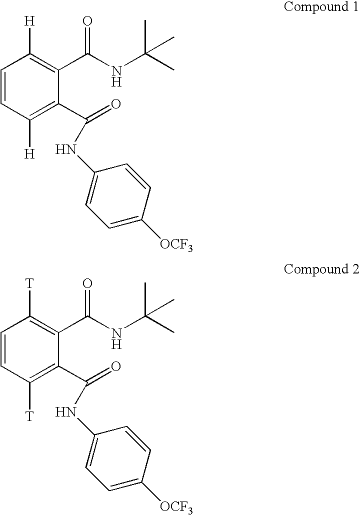

The compound shown in this example is labelled by standard methods known from the literature. In accordance with this method, the unlabelled starting compound (compound 1) is tritiated with the aid of the catalyst (1,5-cyclo-octadiene)(pyridin)(tricyclohexylphosphine)iridium(I) hexafluorophosphate. The starting material (compound 1) was dissolved in dichloromethane and tritiated with constant stirring in the presence of a tritium atmosphere. The catalyst was subsequently removed by HPLC. This gave the compound 2, which was employed as labelled ligand in the binding experiments of Example 2.

The insecticidal activity of the tritiated Compound 2 was verified in the biological test described hereinbelow. Its insecticidal activity did not differ from that of the unlabelled starting compound (Compound 1).

Spodoptera frugiperda test

| | |
|---|---|
| Solvent: | 7 parts by weight of dimethylformamide |
| Emulsifier: | 2 parts by weight of alkylaryl polyglycol ether |

To produce a suitable active compound preparation, 1 part by weight of active compound is mixed with the stated amounts of solvent and emulsifier, and the concentrate is diluted to the desired concentration with emulsifier-containing water.

Cabbage leaves (Brassica oleracea) are treated by being dipped into the active compound preparation of the desired concentration and populated with armyworm (Spodoptera frugiperda) caterpillars while the leaves are still moist. After the desired time, the destruction is determined in %.

### Example 2

### Binding Assay on Insect Membrane Preparations for Identifying Pesticidal Active Compounds

The following text is a description of a method for determining the interaction of test substances with the binding site of the compounds of the general Formula (I) in insect membranes. The radioligand binding method is automatable and suitable for screening substances by the high-throughput method.

1. Preparation of Microsomal Membranes

1.1 Biological Material:

1.1.1 Heliothis virescens L5 Instars, Adult Heliothis virescens, Adult Musca domestica.

1.1.2 CHO Cells Which are Transiently or Stably Transfected with a cDNA of the Drosophila melanogaster CG8272-RA Gene.

Buffers and Reagents:

Buffer 1: 25 mM K-PIPES, pH 7.4, 0.3 M sucrose, 0.2 M KCl, 0.1 mM EGTA, aliquot of a commercial protease inhibitor cocktail.

Buffer 2: 20 mM Tris/HCl, pH 7.4, 0.3 M sucrose, 1.0 mM dithiothreitol, 0.8% (w/v) bovine serum albumin.

1.3 Preparation:

1.3.1 Insect Tissue:

Larvae or the isolated thoraces of adult insects were homogenized in buffer 1 (10 ml g⁻¹ biological material) by means of an Ultra-Turrax. The homogenate was filtered through 2 layers of Miracloth gauze and centrifuged for 10 minutes at 500×g and 4° C. The supernatant was again filtered through 4 layers of Miracloth gauze and subsequently centrifuged for 20 minutes at 8000×g and 4° C. Finally, the supernatant was ultracentrifuged for 60 minutes at 100 000×g and 4° C. to sediment the microsomal membranes.

The sediment was resuspended in a small volume of buffer 2 by means of a Potter homogenizer, and the protein content was brought to 10-20 mg ml⁻¹ by dilution. Aliquots were frozen in liquid nitrogen and stored at −80° C. Membranes of transfected cells:

Transfected CHO cells were suspended in buffer 2 and lysed. The lysate was first centrifuged for 5 minutes at 500×g and 4° C. The cell-free supernatant was centrifuged for 60 minutes at 100 000×g and 4° C. to sediment the cell membranes.

The sediment was resuspended in a small volume of buffer 2 by means of a Potter homogenizer, and the protein content was brought to 5-10 mg ml⁻¹ by dilution. Aliquots were frozen in liquid nitrogen and stored at −80° C.

2. Binding Assay:

2.1 Buffers and Reagents:

Buffer 3: 10 mM HEPES, pH 7.4, 800 μM CaCl₂×2 H₂0, 10 mM ATP, 1.5 M KCl, 0.05% bovine serum albumin.

Buffer 4: 10 mM HEPES, pH 7.4, 150 mM KCl, precooled to 4° C.

2.2 Carrying Out the Assay:

Microtiter plates (96 cavities) were filled with in each case 50 μl/cavity of test substance solutions of suitable concentrations (in water/1% DMSO end concentration, negative controls 1% DMSO, if desired reference compounds as positive controls).

In each case 100 μl of protein solution (diluted in buffer 3, end concentration: 50 μg protein/assay mixture) and 100 μl of radioligand solution (diluted in buffer 3, end concentration around the dissociation constant of the ligand: typically 2-5 nM) were added.

The assay plates were incubated at 22° C. for exactly 2 hours. The assay mixtures were subsequently filtered through GF/B filters (moistened with 0.1% polyethyleneimine) and the filters were washed with 2.0 ml of buffer 4. The filters were treated with scintillator solution and the bound radioactivity was measured in the liquid scintillation counter.

The specific binding is the difference between the measured total radioactivity of the assay mixtures (Bₜₒₜₐₗ) and the bound radioactivity of control mixtures in which the radioligand had been quantitatively displaced from its specific binding site by an at least 10⁴-fold excess of a suitable unlabelled ligand (=B_{unspecific}). The unspecific binding typically amounts to 5 to 25% of the total binding, the maximum specific binding amounts to between 1 and 3 pmol mg⁻¹ of the membrane protein employed.

A reduction of the specific radioligand binding as related to the concentration serves to identify active test substances.

When using the method in the high-throughput screening of chemical substance libraries, the test compounds are assayed individually or as a mixture of 8-12 individual compounds at a specified concentration, for example 10 μM. The significant reduction of the binding of the radioligand in comparison with negative controls (>30%) serves to identify active test compounds which interact specifically with the ligand binding sites.

### Example 3

Inhibition of the Binding of a Labelled Compound of the Formula (I) (Compound 2) by a Test Compound (Compound 1) as Related to Concentration

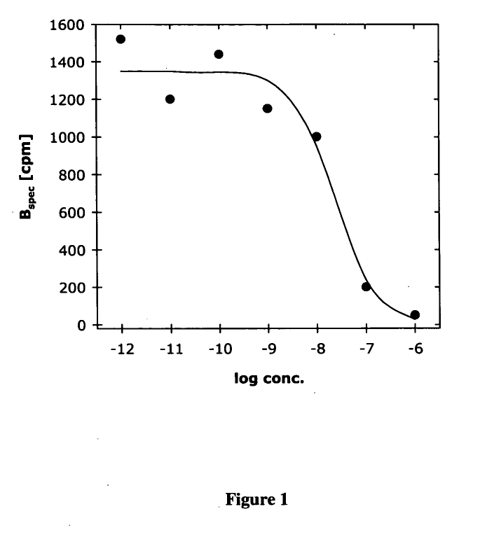

FIG. 1 shows the inhibition of the specific binding of Compound 1 by the test substance (Compound 2) as a function of concentration.

The point of inflection of the sigmoidal curve fitted to the data points (IC₅₀) describes the substance concentration at which 50% of the specific ligand binding are inhibited: IC₅₀=25.1 nM (r²=0.957).

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

1. A method for identifying pesticidally active substances comprising measuring the displacement of a i) labelled compound as shown in the Formula (I) hereinbelow or ii) of a labelled compound which binds to the same binding site as a compound of the Formula (I) on membrane preparations or cell lines containing the molecular target of the compounds of the Formula (I) 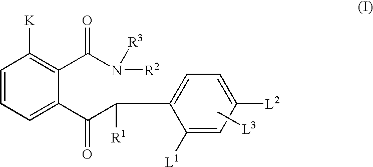
in which
K represents halogen, cyano, alkyl, halogenoalkyl, alkoxy or halogenoalkoxy,
R¹, R², R³ in each case independently of one another represent hydrogen, cyano, C₃-C₈-cycloalkyl which is optionally substituted by halogen, or a group of the formula M¹-Qₖ in which
M¹ represents optionally substituted alkylene, alkenylene or alkynylene,
Q represents hydrogen, halogen, cyano, nitro, halogenoalkyl, in each case optionally substituted C₃-C₈-cycloalkyl, alkylcarbonyl or alkoxycarbonyl, in each case optionally substituted phenyl, hetaryl or a group T-R⁴ in which
T represents —O—, —S(O)ₘ— or 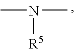
R⁴ represents hydrogen, in each case optionally substituted alkyl, alkenyl, alkynyl, cycloalkyl, cycloalkyl-alkyl, alkoxyalkyl, alkylcarbonyl, alkoxycarbonyl, phenyl, phenyl alkyl, phenylalkoxy, hetaryl, hetarylalkyl,
R⁵ represents hydrogen, in each case optionally substituted alkylcarbonyl, alkoxycarbonyl, phenylcarbonyl or phenylalkoxycarbonyl,
k represents the numbers 1 to 4,
m represents the numbers 0 to 2, or
R¹ and R² together form optionally substituted four- to seven-membered rings which can optionally be interrupted by hetero atoms,
L¹ and L³ independently of one another represent hydrogen, halogen, cyano or in each case optionally substituted alkyl, alkoxy, alk-S(O)ₘ—, phenyl, phenoxy or hetaryloxy,
L² represents hydrogen, halogen, cyano, in each case optionally substituted alkyl, alkenyl, alkynyl, halogenoalkyl, cycloalkyl, phenyl, hetaryl or the group M²-R⁶ in which
M² represents —O— or —S(O)ₘ—,
R⁶ represents in each case optionally substituted alkyl, alkenyl, alkynyl, cycloalkyl, phenyl or hetaryl, or
L¹ and L³ or L¹ and L² optionally together form an optionally substituted five- to six-membered ring which can optionally be interrupted by hetero atoms.

2. The method according to claim 1 wherein
K represents fluorine, chlorine, bromine, iodine, cyano, C₁-C₆-alkyl, C₁-C₆-halogenoalkyl, C₁-C₆-alkoxy or C₁-C₆-halogenoalkoxy.
R¹, R², R³ in each case independently of one another represent hydrogen, cyano, C₃-C₆-cycloalkyl which is optionally substituted by halogen, or a group of the formula M¹-Qₖ, in which
M¹ represents C₁-C₈-alkylene, C₃-C₆-alkenylene or C₃-C₆-alkynylene,
Q represents hydrogen, halogen, cyano, nitro, halogenoalkyl, or represents C₃-C₈-cycloalkyl which is optionally substituted by fluorine, chlorine, C₁-C₆-alkyl or C₁-C₆-alkoxy and in which one or two ring members which are not directly adjacent are optionally replaced by oxygen and/or sulphur, or represents C₁-C₆-alkylcarbonyl or C₁-C₆-alkoxycarbonyl, each of which is optionally substituted by halogen, or represents phenyl or hetaryl having five to six ring atoms, each of these phenyl or hetaryl rings optionally being substituted by halogen, C₁-C₆-alkyl, C₁-C₆-halogenoalkyl, C₁-C₆-alkoxy, C₁-C₆-halogenoalkoxy, cyano or nitro, or represents a group T-R⁴ in which
T represents —O—, —S(O)ₘ— or 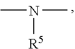
R⁴ represents hydrogen, or represents C₁-C₈-alkyl, C₃-C₈-alkenyl, C₃-C₈-alkynyl, C₃-C₈-cycloalkyl, C₃-C₈-cycloalkyl-C₁-C₂-alkyl, C₁-C₆-alkylcarbonyl, C₁-C₆-alkoxycarbonyl, each of which is optionally substituted by fluorine and/or chlorine, or represents phenyl, C₁-C₄-phenylalkyl, C₁-C₄-phenylalkyloxy, hetaryl or hetarylalkyl, each of which is optionally monosubstituted to tetrasubstituted by halogen, C₁-C₆-alkyl, C₁-C₆-alkoxy, C₁-C₄-halogenoalkyl, C₁-C₄-halogenoalkoxy, nitro or cyano, hetaryl encompassing 5 to 6 ring atoms,
R⁵ represents hydrogen, or represents C₁-C₆-alkylcarbonyl, C₁-C₆-alkoxycarbonyl, each of which is optionally substituted by fluorine and/or chlorine, or represents phenylcarbonyl or phenyl-C₁-C₄-alkyloxycarbonyl, each of which is optionally monosubstituted to tetrasubstituted by halogen, C₁-C₆-alkyl, C₁-C₆-alkoxy, C₁-C₄-halogenoalkyl, C₁-C₄-halogenoalkoxy, nitro or cyano,
k represents the numbers 1 to 3
m represents the numbers 0 to 2, or
R¹ and R² form a five- to six-membered ring which can optionally be interrupted by an oxygen or sulphur atom,
L¹ and L³ independently of one another represent hydrogen, fluorine, chlorine, bromine, iodine, cyano, C₁-C₆-alkyl, C₁-C₄-halogenoalkyl, C₁-C₆-alkoxy, C₁-C₄-halogenoalkoxy, C₁-C₄-alkyl-S(O)ₘ—, C₁-C₄-haloalkyl-S(O)ₘ—, or represent phenyl, phenoxy, pyridyloxy, thiazolyloxy or pyrimidyloxy, each of which is optionally monosubstituted to trisubstituted by fluorine, chlorine, bromine, C₁-C₆-alkyl, C₁-C₆-alkoxy, C₁-C₄-halogenoalkyl, C₁-C₄-halogenoalkoxy, cyano or nitro,
L² represents hydrogen, fluorine, chlorine, bromine, iodine, cyano, or represents C₁-C₁₀-alkyl, C₂-C₁₀-alkenyl, C₂-C₆-alkynyl, each of which is optionally substituted by fluorine and/or chlorine, or represents C₃-C₆-cycloalkyl, which is optionally substituted by fluorine or chlorine, or represents phenyl, pyridyl, thienyl, pyrimidyl or thiazolyl, each of which is optionally monosubstituted to trisubstituted by fluorine, chlorine, bromine, C₁-C₆-alkyl, C₁-C₆-alkoxy, C₁-C₄-halogenoalkyl, C₁-C₄-halogenoalkoxy, cyano or nitro, or represents a group M²-R⁶ in which
M² represents —O— or —S(O)ₘ— and
R⁶ represents C₁-C₈-alkyl, C₂-C₈-alkenyl, C₃-C₆-alkynyl or C₃-C₆-cycloalkyl, each of which is optionally substituted by fluorine and/or chlorine, or represents phenyl, pyridyl, pyrimidyl or thiazolyl, each of which is optionally monosubstituted to trisubstituted by fluorine, chlorine, bromine, C₁-C₆-alkyl, C₁-C₆-alkoxy, C₁-C₄-halogenoalkyl, C₁-C₄-halogenoalkoxy, cyano or nitro, or
L¹ and L³ or L² and L³ together in each case form a five- to six-membered ring which is optionally substituted by fluorine and/or C₁-C₂-alkyl and which can optionally be interrupted by one or two oxygen atoms.

3. The method according to claim 1 wherein
K represents chlorine, bromine or iodine,
R¹, R², R³ in each case independently of one another represent hydrogen or a group of the formula M¹-Qₖ, in which
M¹ represents C₁-C₈-alkylene, C₃-C₆-alkenylene or C₃-C₆-alkynylene,
Q represents hydrogen, fluorine, chlorine, cyano, trifluoromethyl, C₃-C₆-cycloalkyl or a group T-R⁴ in which
T represents —O— or —S(O)ₘ—,
R⁴ represents hydrogen, or represents C₁-C₆-alkyl, C₃-C₆-alkenyl, C₃-C₆-alkynyl or C₃-C₆-cycloalkyl, each of which is optionally monosubstituted to trisubstituted by fluorine and/or chlorine,
k represents the numbers 1 to 3
m represents the numbers 0 to 2,
L¹ and L³ independently of one another represent hydrogen, fluorine, chlorine, bromine, iodine, cyano, C₁-C₄-alkyl, C₁-C₂-halogenoalkyl, C₁-C₄-alkoxy, C₁-C₂-halogenoalkoxy, or represent phenyl or phenoxy, each of which is optionally monosubstituted or disubstituted by fluorine, chlorine, bromine, C₁-C₄-alkyl, C₁-C₄-alkoxy, C₁-C₂-halogenoalkyl, C₁-C₂-halogenoalkoxy, cyano or nitro,
L² represents hydrogen, fluorine, chlorine, bromine, iodine, cyano, or represents C₁-C₆-alkyl, C₂-C₆-alkenyl, C₂-C₆-alkynyl, C₃-C₆-cycloalkyl, each of which is optionally monosubstituted to tridecasubstituted by fluorine and/or chlorine, or represents a group M²-R⁶ in which
M² represents —O— or —S(O)ₘ— and
R⁶ represents C₁-C₆-alkyl, C₂-C₆-alkenyl, C₂-C₆-alkynyl or C₃-C₆-cycloalkyl, each of which is optionally monosubstituted to tridecasubstituted by fluorine and/or chlorine, or represents phenyl or pyridyl, each of which is optionally monosubstituted to disubstituted by fluorine, chlorine, bromine, C₁-C₄-alkyl, C₁-C₄-alkoxy, trifluoromethyl, difluoromethoxy, trifluoromethoxy, cyano or nitro.

4. The method according to claim 1 wherein the compound of the Formula (I) is the following compound:

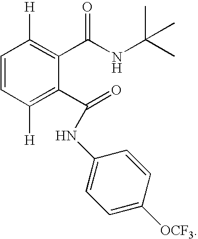

5. The method according to claim 1 wherein the compound of the Formula (I) is labelled with a radioactive isotope.

6. The method according to claim 5 wherein the compound of Formula (I) is tritiated.

7. The method according to claim 1 further comprising:
a1) producing a membrane preparation starting from adult organisms or their larvae, or
a2) producing a membrane preparation starting from cells which express the molecular target of the compounds of the Formula (I),
b) preparing a protein solution starting from said membrane preparations,
c) contacting the protein solution with a labelled compound of the Formula (I) under conditions which permit the binding of the labelled compound to the molecular target,
d) contacting the mixture prepared in step c) with an unlabelled test substance,
e) measuring if a displacement of the labelled compound by the test substance has taken place, and, if appropriate,
f) testing the test substance for pesticidal properties.

8. The method according to claim 7 wherein in step a1), membrane preparations are produced starting from adult insects of the species Musca domestica.

9. The method according to claim 7 wherein in step a1), membrane preparations are produced starting from adult insects of the species Heliothis virescens or their larvae.
